(19) United States
(12) Patent Application Publication
(10) Pub. No.: US 2025/0017721 A1
(43) Pub. Date: Jan. 16, 2025

(54) BREAST TREATMENT DEVICE

(71) Applicant: LifeCell Corporation, North Chicago, IL (US)

(72) Inventors: Aaron M. Barere, Hoboken, NJ (US); Sangwook Park, Virginia Beach, VA (US); Evan J. Friedman, West Orange, NJ (US); Kai-Roy Wang, Jersey City, NJ (US)

(21) Appl. No.: 18/668,494

(22) Filed: May 20, 2024

Related U.S. Application Data
(63) Continuation of application No. 17/069,507, filed on Oct. 13, 2020, now abandoned.
Application No. 17/069,507 is a continuation of application No. 15/241,388, filed on Aug. 19, 2016, now Pat. No. 10,842,612.
(60) Provisional application No. 62/208,366, filed on Aug. 21, 2015.

(51) Int. Cl. A61F 2/12; A61L 27/36

(52) U.S. Cl. CPC A61F 2/12; A61L 27/362; A61L 27/3633; A61L 27/3695; A61F 2230/0013

(57) ABSTRACT

The present disclosure provides devices and methods for treating the breast. The devices can include an acellular tissue matrix having a predefined shape. The shape can include a first edge with an S-shaped configuration and a second arcuate-shaped edge. The shape alternatively can include a first concave edge and a second convex edge.

This application is a continuation of U.S. patent application Ser. No. 17/069,507, filed Oct. 13, 2020, which is a continuation of U.S. patent application Ser. No. 15/241,388, filed Aug. 19, 2016, now U.S. Pat. No. 10,842,612, issued on Nov. 24, 2020, which claims the benefit, under 35 U.S.C. § 119(e), of U.S. Provisional Patent Application No. 62/208,366, entitled “Breast Treatment Device,” filed Aug. 21, 2015. Each of the above referenced applications incorporated herein by reference in their entirety.

The present disclosure relates generally to devices for improving breast surgeries, including tissue matrices specially shaped and sized for breast reconstruction or augmentation.

### BACKGROUND

The use of acellular tissue matrices such as ALLODERM®, a dermal acellular matrix produced by LIFECELL® CORPORATION (Branchburg, NJ)), for use in breast procedures has become increasingly popular with plastic surgeons. Such materials provide a number of advantages and can be used to replace or augment supportive structures after, for example, mastectomy. Such materials can also be useful in aesthetic procedures (e.g., breast augmentation) by providing additional support for breast implants, allowing improved control of breast shape, preventing skin rippling, and/or preventing or treating other problems that may occur with breast augmentation (e.g., symmastia and bottoming out.)

For many surgical procedures, in order to achieve desired results, positioning an implant with respect to anatomical structure and other medical devices is important. To improve both surgical results and efficiency, pre-sized and pre-shaped tissue matrices can be beneficial. The present application provides improved breast treatment devices including tissue matrix materials specially shaped and sized to improve surgical breast procedures.

Accordingly, in some embodiments, a breast treatment device is provided. The device can include a sheet of acellular tissue matrix, wherein the sheet of acellular tissue matrix comprises a flexible sheet with a top surface, a bottom surface, and a peripheral border. In one embodiment, the peripheral border comprises a first edge having a substantially S-shaped configuration, and an arcuate-shaped second edge. In another embodiment, the peripheral border comprises a first edge having a concave shape, and a convex-shaped second edge.

Also provided is a breast treatment system including a breast implant or tissue expander. The system can also include a sheet of acellular tissue matrix, wherein the sheet of acellular tissue matrix comprises a flexible sheet with a top surface, a bottom surface, and a peripheral border. In one embodiment, the peripheral border comprises a first edge having a substantially S-shaped configuration, and an arcuate-shaped second edge. In another embodiment, the peripheral border comprises a first edge having a concave shape, and a convex-shaped second edge.

Also provided are methods of treating a breast. The methods can include identifying an anatomic site within a breast and selecting a breast treatment device comprising a sheet of acellular tissue matrix. The methods can further include implanting the treatment device in or proximate the breast and securing at least a portion of the treatment device to tissue in or near the breast. The sheet of acellular tissue matrix can comprise a flexible sheet with a top surface, a bottom surface, and a peripheral border. In one embodiment, the peripheral border comprises a first edge having a substantially S-shaped configuration, and an arcuate-shaped second edge. In another embodiment, the peripheral border comprises a first edge having a concave shape, and a convex-shaped second edge. The method can include securing at least a portion of the second edge and/or additional parts of the tissue matrix to tissue in or near the breast.

### BRIEF DESCRIPTION OF THE DRAWINGS

Reference will now be made to exemplary embodiments, examples of which are illustrated in the accompanying drawings. Wherever possible, the same reference numbers will be used throughout the drawings to refer to the same or like parts. The drawings are not necessarily to scale.

FIG. 1 illustrates a breast treatment device, including a pre-shaped tissue matrix, according to certain embodiments.

FIG. 2 illustrates a perspective view of a breast treatment device, including a pre-shaped tissue matrix, according to certain embodiments.

FIG. 3 illustrates implantation of a system for surgical breast procedures, including a pre-shaped tissue matrix implanted along with a breast implant or tissue-expander, according to certain embodiments.

FIG. 4 illustrates a breast treatment device, including a pre-shaped tissue matrix, according to other embodiments.

FIG. 5 illustrates a breast treatment device, including a pre-shaped tissue matrix, according to other embodiments.

FIG. 6 illustrates a breast treatment device, including a pre-shaped tissue matrix, according to other embodiments.

FIG. 7 illustrates implantation of a system for surgical breast procedures, including a pre-shaped tissue matrix implanted along with a breast implant or tissue-expander, according to certain embodiments.

### DESCRIPTION OF EXEMPLARY EMBODIMENTS

Reference will now be made in detail to various embodiments of the disclosed devices and methods, examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Any range described herein will be understood to include the endpoints and all values between the endpoints.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

The present disclosure relates generally to devices for surgical breast procedures and systems and methods relating to such devices. The devices can be used for tissue augmentation, repair or regeneration of damaged tissue, and/or correction of tissue defects. As such, the devices, systems, and methods discussed herein can be suitable for a wide range of surgical applications, such as, for example, aesthetic surgery, breast reconstruction, breast augmentation, breast enhancement, breast reduction, and revisionary breast surgeries.

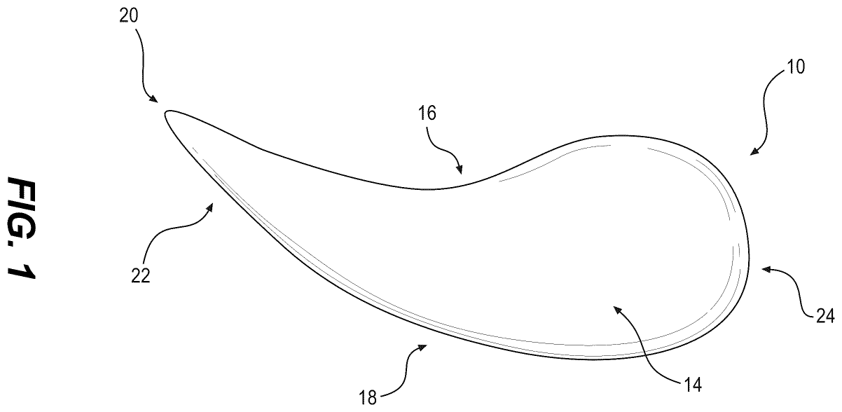

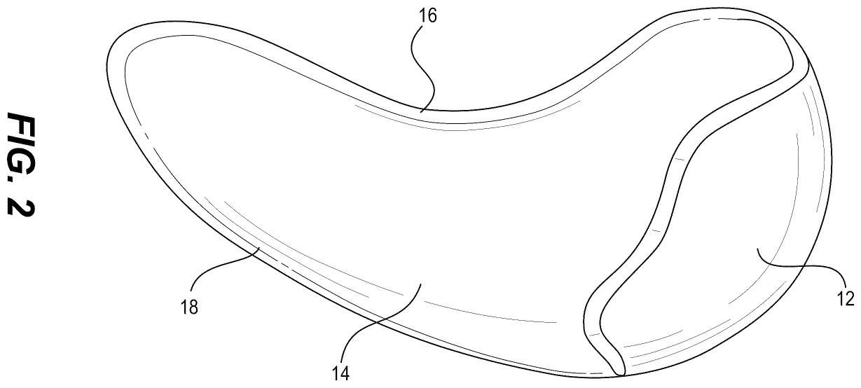

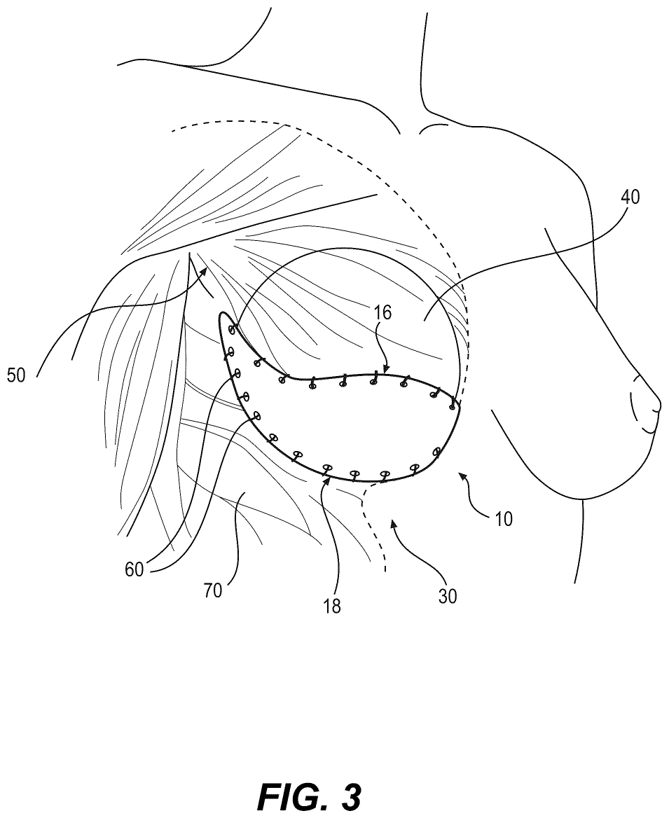

FIG. 1 illustrates a breast treatment device 10, including a pre-shaped tissue matrix, according to certain embodiments, and FIG. 2 illustrates a perspective view of the device 10 of FIG. 1. The device 10 can include a sheet of acellular tissue matrix. The sheet can be a flexible material with a top surface 14, a bottom surface 12 (FIG. 2), and a peripheral border. The peripheral border can include at least two edges 16, 18, including a first edge 16 having a substantially S-shaped configuration, and an arcuate-shaped second edge 18. As discussed below, the device 10 can form part of a treatment system 30, including a breast implant or tissue expander (FIG. 3).

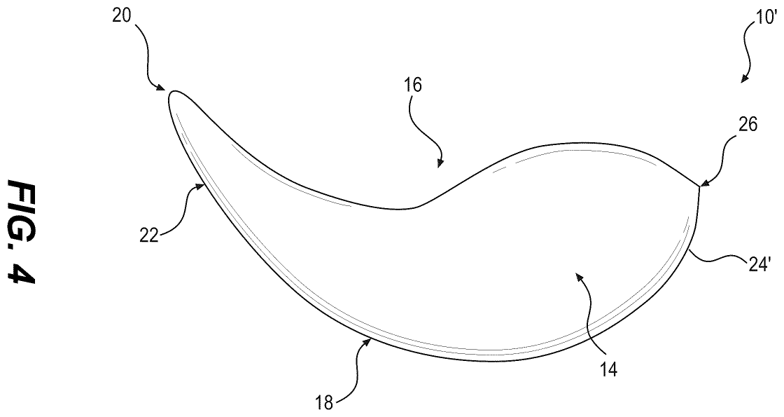

The first edge 16 and second edge 18 can be joined to one another in a variety of configurations. For example, in one embodiment the first edge 16 and second edge 18 meet at a first end 22 of the device 10 and at a second end 24 of the device 10. Furthermore, the shape of the device at the first end 22 and/or second end 24 can have a variety of configurations, including a pointed tip 20, a rounded shape (FIGS. 1 and 2 at second end 24)), a curvilinear shape, or any other suitable configuration. In one embodiment, both the first end 22 and second end 24 have a pointed tip 20, 26. (FIG. 4).

As noted above, the devices discussed herein can be used for treatment of a breast. Accordingly, the devices 10 can be part of a system 30 for treating a breast-the system 30 including a device 10 and an implant such as a breast implant or breast tissue expander 40. A variety of suitable implants (e.g., saline filled breast implants) and tissue expanders can be used.

The tissue matrices used to produce the devices described herein can include a variety of different materials. For example, an acellular tissue matrix or other tissue product can be selected to allow tissue ingrowth and remodeling to assist in regeneration of tissue normally found at the site where the matrix is implanted. For example, an acellular tissue matrix, when implanted on or into subdermal tissue, fascia, mammary tissue, or other tissue, may be selected to allow regeneration of the tissue without excessive fibrosis or scar formation. In certain embodiments, the devices can be formed from ALLODERM® or STRATTICE™ (LIFECELL® CORPORATION, BRANCHBURG, NJ) which are human and porcine acellular dermal matrices, respectively. Alternatively, other suitable acellular tissue matrices can be used. For example, a number of biological scaffold materials as described by Badylak et al., or any other similar materials, can be used to produce tissues with a stable three-dimensional shape. Badylak et al., “Extracellular Matrix as a Biological Scaffold Material: Structure and Function,” Acta Biomaterialia (2008), doi:10.1016/j.actbio.2008.09.013. The devices described herein can be produced from a variety of different human or animal tissues including human, porcine, ovine, bovine, or other animal tissues.

In some cases, the tissue matrices can be produced from materials that include a basement membrane on at least one surface. For example, the devices can be produced from an acellular dermal matrix, and either the top surface 14 or bottom surface 12 can include an epithelial basement membrane across the surface. During implantation, the device 10 should generally be positioned such that the basement membrane surface is positioned facing the most vascular tissue. For example, as discussed below, when implanted next to a breast implant or tissue expander, the basement membrane covered surface may face towards the implant or tissue expander such that the surface not including a basement membrane faces overlying vascularized tissue.

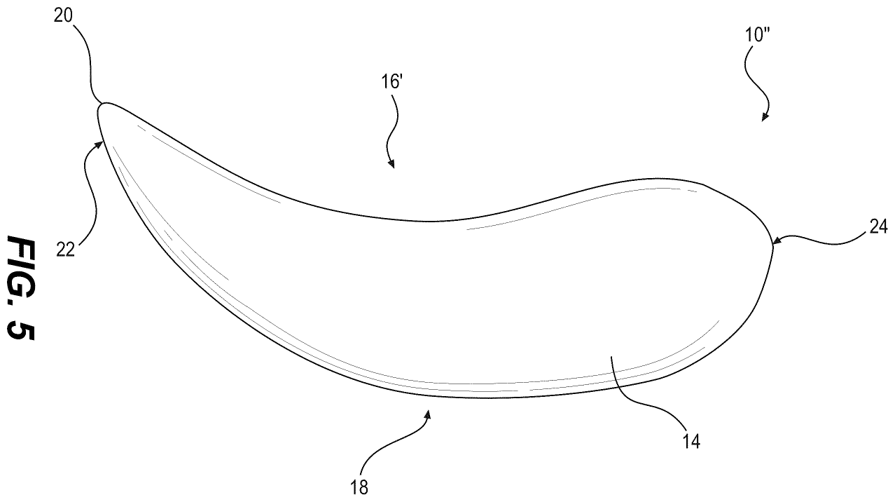

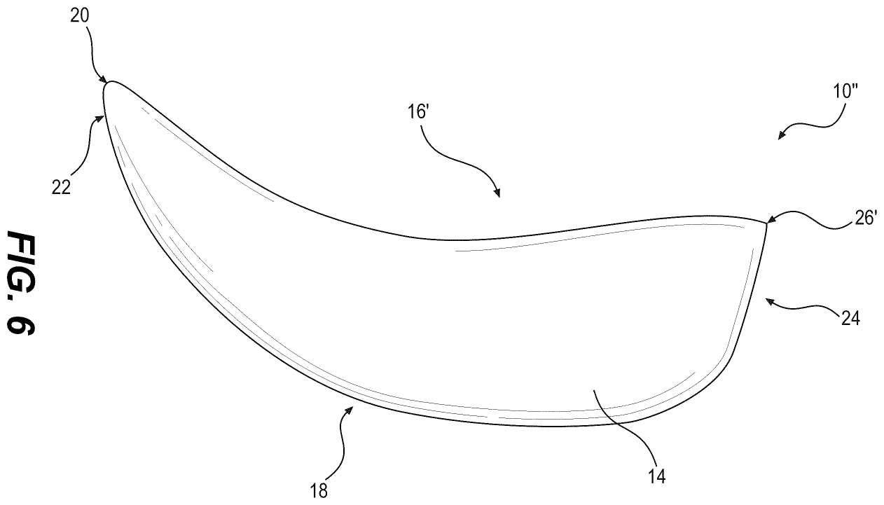

The devices and systems disclosed herein can also have other shapes and configurations. For example, FIG. 5 illustrates a perspective view of a breast treatment device 10″, including a pre-shaped tissue matrix, according to other embodiments. FIG. 6 illustrates a perspective view of a variation of the device 10″ shown in FIG. 5.

As shown, the device 10″ includes a sheet of acellular tissue matrix. And similar to FIGS. 1, 2, and 4, the sheet of acellular tissue matrix comprises a flexible sheet with a top surface 14 and a bottom surface 12 (not shown but reflected on back side of device 10″). The device 10″ also includes a peripheral border, wherein the peripheral border comprises a first edge 16′ having a concave shape, and a convex-shaped second edge 18.

Similar to the devices discussed above, the device 10″ can include a first end 22 and second end 24, and the first edge 16′ and second edge 18 can be joined at the first end 22 and second end 24. In one embodiment, the first end 22 includes a pointed tip 20, and the second end 24 includes a rounded end. In another embodiment, the first end 22 includes a pointed tip 20, and the second end 24 includes a pointed tip 26′ (FIG. 6). Furthermore, the devices shown in FIG. 5-6 can be formed of the materials discussed above, with or without a basement membrane.

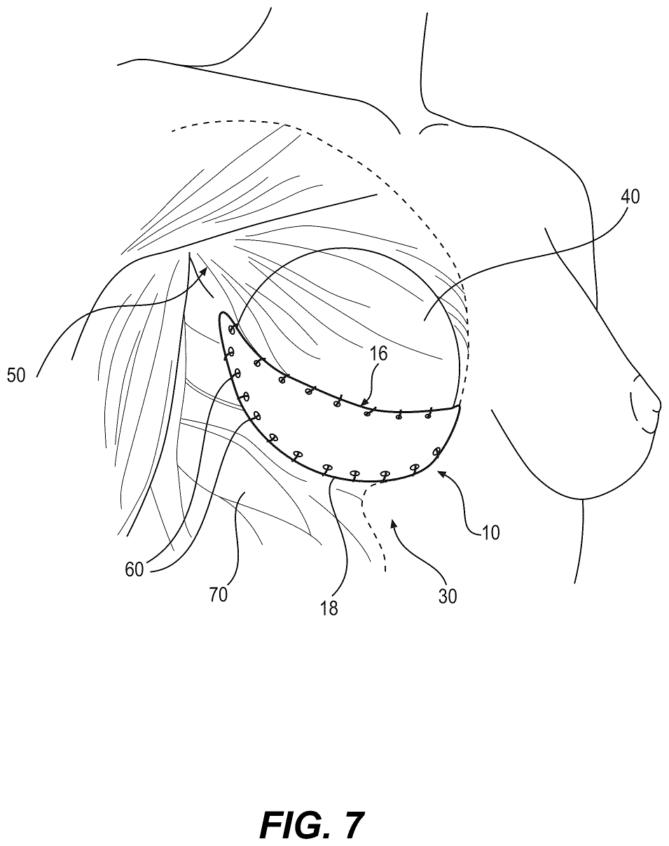

Also disclosed herein are methods for treating a breast. Accordingly, FIG. 3 and FIG. 7 illustrate implantation of a system for surgical breast procedures, including a pre-shaped tissue matrix implanted with a breast implant or tissue-expander, according to certain embodiments. The method can first include identifying an anatomic site within a breast. (As used herein, “within a breast” will be understood to be within mammary tissue, or within or near tissue surrounding the breast such as tissue just below, lateral or medial to the breast, or beneath or within surrounding tissues including, for example, under chest (pectoralis) muscles 50, and will also include implantation in a site in which part or all of the breast has already been removed via a surgical procedure). The site can include, for example, any suitable site needing reconstruction, repair, augmentation, or treatment. Such sites may include sites in which surgical oncology procedures (mastectomy, lumpectomy) have been performed, sites where aesthetic procedures are performed (augmentation or revisions augmentation), or sites needing treatment due to disease or trauma.

After selection of the site, a treatment device is selected. As noted above, various devices including acellular tissue matrices can be used, and the devices can include a flexible sheet having a top surface, a bottom surface, and a peripheral border. The peripheral border and shape of the devices can include any configuration discussed herein.

The method can also include securing at least a portion of the device to a patient. For example, in one embodiment, a portion of the device is secured to a chest wall 70, to surrounding fascia, or to part of an implant or tissue expander 40. In one embodiment, the at least a portion of the second edge 18 is secured to tissue using, for example, suture 60, or other suitable attachments. In addition, other portions of the device 10, including portions of the first edge 16, can be secured to tissue, or if appropriate, to the implant or tissue expander 40 (e.g., via surface features on a tissue expander.)

The methods disclosed herein can also include implantation of an implant or tissue expander 40 under or near part of the device 10, 10′, 10″. In some cases, no implant or expander will be used, but the device 10, 10′, 10″ will be implanted to provide added tissue, e.g., for incision closure after mastectomy. In other cases, the implant or expander 40 will be implanted at the same time as the device 10, 10′, 10″, or in a subsequent surgical procedure.

In addition, FIG. 3 and FIG. 7 illustrate implantation of a device in a right breast, but it will be appreciated that similar devices can be used for treatment of the left breast using a device having a mirror image to that of the devices illustrated in FIG. 3 and FIG. 7. Further, if a basement membrane is included, the basement membrane may similarly be positioned to face a tissue expander or implant, as discussed above.

Other embodiments will be apparent to those skilled in the art from consideration of the specification and practice of this disclosure. It is intended that the specification and examples be considered as exemplary only, with the true scope and spirit of the disclosed devices and methods being indicated by the following claims.

1. A breast treatment device, comprising:
a sheet of acellular tissue matrix, wherein the sheet of acellular tissue matrix comprises a flexible sheet with a top surface, a bottom surface, and a peripheral border, and wherein the peripheral border comprises a first edge having a concave shape, and a convex-shaped second edge.

2. The device of claim 1, wherein the first edge and second edge meet at a first end of the device and at a second end of the device.

3. The device of claim 2, wherein the first end of the device comprises a first pointed tip.

4. The device of claim 3, wherein the second end of the device comprises a second pointed tip.

5. The device of claim 2, wherein the second end of the device comprises a rounded edge.

6. (canceled)

7. The device of claim 1, wherein the tissue matrix comprises a tissue matrix derived from porcine tissue.

8. The device of claim 1, wherein the tissue matrix comprises a dermal tissue matrix.

9. (canceled)

10. A method of treating a breast, comprising:
identifying an anatomic site within a breast;
selecting a breast treatment device comprising a sheet of acellular tissue matrix, wherein the sheet of acellular tissue matrix comprises a flexible sheet with a top surface, a bottom surface, and a peripheral border, and wherein the peripheral border comprises a first edge having a concave shape, and a convex-shaped second edge; and
implanting the treatment device in or proximate the breast; and
securing at least a portion of the treatment device along the second convex-shaped edge to tissue in or near the breast.

11. The method of claim 10, further comprising implanting a breast implant or tissue expander within the breast.

12. The method of claim 11, wherein the breast implant or tissue expander comprises a silicone or saline filled implant or tissue expander.

13. The method of claim 10, wherein the treatment device is implant with the convex-shaped second edge facing inferiorly.

14. (canceled)

15. The method of claim 10, wherein the tissue matrix comprises a tissue matrix derived from porcine tissue.

16. The method of claim 10, wherein the tissue matrix comprises a dermal tissue matrix.

17. (canceled)

18. (canceled)

19. A breast treatment system, comprising:
a breast implant or tissue expander; and
a sheet of acellular tissue matrix, wherein the sheet of acellular tissue matrix comprises a flexible sheet with a top surface, a bottom surface, and a peripheral border, and wherein the peripheral border comprises a first edge having a concave shape, and a convex-shaped second edge.

20. The breast treatment system of claim 19, wherein the first edge and second edge meet at a first end of the device and at a second end of the device.

21. The breast treatment system of claim 20, wherein the first end of the device comprises a first pointed tip.

22. The breast treatment system claim 21, wherein the second end of the device comprises a second pointed tip.

23. The breast treatment system of claim 20, wherein the second end of the device comprises a rounded edge.

24. (canceled)

25. The breast treatment system of claim 19, wherein the tissue matrix comprises a tissue matrix derived from porcine tissue.

26. The breast treatment system of claim 19, wherein the tissue matrix comprises a dermal tissue matrix.

27. (canceled)
